(19) United States
(12) Patent Application Publication
(10) Pub. No.: US 2007/0249536 A1
(43) Pub. Date: Oct. 25, 2007

(54) THREE-COMPONENT POLYANHYDRIDE COPOLYMERS AND A METHOD OF FORMING THE SAME

(76) Inventors: Peter X. Ma, Ann Arbor, MI (US); Sijian Hou, Tucker, GA (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 11/739,757

(22) Filed: Apr. 25, 2007

Related U.S. Application Data
(60) Provisional application No. 60/794,617, filed on Apr. 25, 2006.

(51) Int. Cl. A61K 38/00

(52) U.S. Cl. 514/12

(57) ABSTRACT

A three-component polyanhydride copolymer having tunable erosion properties includes a sebacic acid anhydride precursor, a 1,3-bis(carboxyphenoxy) propane anhydride precursor, and a poly(ethylene glycol) anhydride precursor. The erosion rate of the copolymer increases with an increasing amount of the poly(ethylene glycol) precursor. A method for forming the three-component polyanhydride copolymer includes combining the sebacic acid precursor, the 1,3-bis(carboxyphenoxy) propane precursor, and the polyethylene glycol precursor to form a precursor mixture. The precursor mixture is then melt polymerized to form the three-component polyanhydride copolymer.

### CROSS-REFERENCE TO RELATED APPLICATIONS

This application claims the benefit of U.S. Provisional Patent application Ser. No. 60/794,617 filed on Apr. 25, 2006, incorporated by reference herein in its entirety.

### STATEMENT REGARDING FEDERALLY SPONSORED RESEARCH OR DEVELOPMENT

This invention was made in the course of research partially supported by a grant from the National Aeronautics and Space Administration (NASA) Bioscience and Engineering Institute, Grant No. NNC04AA21A, and the National Institutes of Health (NIH), Grant No. DE015384. The U.S. government has certain rights in the invention.

### BACKGROUND

The present disclosure relates generally to three-component polyanhydride copolymers and a method for forming the same.

Parathyroid hormone (PTH) is a peptide hormone that is capable of exhibiting either anabolic or catabolic effects on bone, depending, at least in part, on the dosage and delivery pattern. Generally, a continuous high dose delivery of PTH leads to catabolic effects, while a continuous low dose or a pulsatile high dose delivery of PTH results in anabolic effects on bone. Daily injections may be an anabolic treatment, however, such treatment is less convenient and may not be favorable to patients. As such, controlled delivery of PTH, and other like substances, in an anabolic fashion is highly desirable.

Biodegradable polymers have been suggested to protect the bioactivity of PTH for controlled release formulations. Biodegradable poly(lactic acid-co-glycolic acid) (PLGA) microspheres may encapsulate PTH to protect it from denaturing and to control the delivery duration through a variety of polymer structures. However, the bulk erosion nature of the PLGA does not allow for the pulsatile release of PTH.

Polyanhydrides (e.g., sebacic acid (SA)-1,3-bis(p-carboxyphenoxy) propane (CPP) polyanhydrides) have been used as surface-erosion matrix materials to achieve zero-order release (i.e., substantially constant release rate) of drugs uniformly distributed in the matrix. The erosion of these polyanhydrides may be complicated, as it may be affected by many factors, such as, for example, the chemical nature of the anhydride bonds, the crystallization of the bulk polymer, and the degradation products. Furthermore, these polyanhydrides are very hydrophobic, and their hydrolytic degradation may take a relatively long time. The slow erosion and dissolution are generally not suitable for pulsatile release profiles.

As such, it would be desirable to provide a biodegradable polymer system with tunable erosion properties to achieve pulsatile release profiles.

### SUMMARY

A three-component polyanhydride copolymer having tunable erosion properties includes a sebacic acid anhydride precursor, a 1,3-bis(carboxyphenoxy) propane anhydride precursor, and a poly(ethylene glycol) anhydride precursor. The erosion rate of the copolymer increases with an increasing amount of the poly(ethylene glycol) precursor.

A method for forming the three-component polyanhydride copolymer includes combining the sebacic acid precursor, the 1,3-bis(carboxyphenoxy) propane precursor, and the polyethylene glycol precursor to form a precursor mixture. The precursor mixture is then melt polymerized to form the three-component polyanhydride copolymer.

### BRIEF DESCRIPTION OF DRAWINGS

Features and advantages of embodiments of the present disclosure will become apparent by reference to the following detailed description and drawings, in which like reference numerals correspond to similar, though not necessarily identical components. For the sake of brevity, reference numerals or features having a previously described function may not necessarily be described in connection with other drawings in which they appear.

FIGS. 1A and 1B are schematic diagrams of the synthesis of a poly(ethylene glycol) anhydride precursor, and of the synthesis of sebacic acid (SA)-1,3-bis(carboxyphenoxy) propane (CPP)-poly(ethylene glycol) (PEG) polyanhydrides, respectively;

FIG. 2 is an ¹H-NMR spectrum of a SA-CPP-PEG polyanhydride;

FIG. 3 is a graph depicting the effects of reaction temperature and reaction time on the molecular weight of polyanhydrides;

FIGS. 4A through 4D are graphs depicting the mass loss profiles (erosion experiments were conducted at 37° C. in 0.1M PBS) of A) SA-CPP-PEG600 polyanhydride with a specimen thickness of 0.5 mm, B) SA-CPP-PEG600 polyanhydride with a specimen thickness of 1 mm, C) SA-CPP polyanhydride discs with a diameter of 3.55 mm and two different thicknesses as indicated, D) SA-CPP-PEG600 (2.6%) polyanhydride discs with a diameter of 3.55 mm and two different thicknesses as indicated;

FIGS. 5A through 5C are graphs depicting a change in solution pH value over time (erosion experiments were conducted at 37° C. in 0.1M PBS) for A) SA-CPP-PEG600 polyanhydrides (½ mm thick), B) SA-CPP-PEG600 polyanhydrides (1 mm thick), C) SA-CPP-PEG1000 polyanhydrides (1 mm thick);

FIGS. 6A and 6B are Scanning Electron Micrograph (SEM) images of an eroding SA-CPP-PEG1000 (1.1%) polyanhydride specimen after one day of erosion at A) 50× magnification and B) 500× magnification; and

FIG. 7 is a graph depicting the thicknesses of residual polyanhydrides after 1 day of erosion, the values represented by bars having an upward slant are the thicknesses as measured using Scanning Electron Microscopy, and the values represented by the bars having a downward slant were calculated from the remaining mass (SA-CPP-PEG1000 polyanhydride cylinder specimens were 1 mm in thickness and 3.55 mm in diameter).

### DETAILED DESCRIPTION

Without being bound to any theory, it is believed that some surface erosion materials may be useful in the fabrication of delivery devices that are capable of achieving a pulsatile release profile. Embodiments of the three-component polyanhydride copolymer disclosed herein incorporate relatively small amounts of poly(ethylene glycol) (PEG) segments into a two-component polyanhydride. It is believed that the three-component polyanhydride copolymers retain the surface erosion characteristics of the two-component polyanhydride, while increasing the erosion rate. The increased erosion rate may be due, at least in part, to an increase in hydrophilicity and/or a decrease in crystallinity of the degradation products. It is further believed that by adjusting the PEG content, relatively faster surface erosion may be achieved. The tunable surface erosion properties of the three-component polyanhydride copolymer disclosed herein may advantageously make the copolymer suitable for fabricating a pulsatile delivery device.

Generally, the copolymer includes a sebacic acid (SA) anhydride precursor, a 1,3-bis(carboxyphenoxy) propane (CPP) anhydride precursor, and a poly(ethylene glycol) (PEG) anhydride precursor. It is believed that the erosion rate and the hydrophilicity of the copolymer increases with an increasing amount of the poly(ethylene glycol) precursor.

In an embodiment, a ratio of the sebacic acid anhydride precursor to the 1,3-bis(carboxyphenoxy) propane anhydride precursor ranges from about 50:50 to about 90:10; and in an alternate embodiment, ranges from about 95:5 to about 5:95. In a further embodiment, the ratio of the sebacic acid anhydride precursor to the 1,3-bis(carboxyphenoxy) propane anhydride precursor is about 80:20. In another embodiment, the three-component polyanhydride copolymer includes the poly(ethylene glycol) anhydride precursor in an amount ranging from about 1% to about 25% with respect to a total molar amount of the sebacic acid precursor and the 1,3-bis(carboxyphenoxy) propane precursor. In an alternate embodiment, the three-component polyanhydride copolymer includes the poly(ethylene glycol) anhydride precursor in an amount ranging from about 1% to about 10% with respect to a total molar amount of the sebacic acid precursor and the 1,3-bis(carboxyphenoxy) propane precursor.

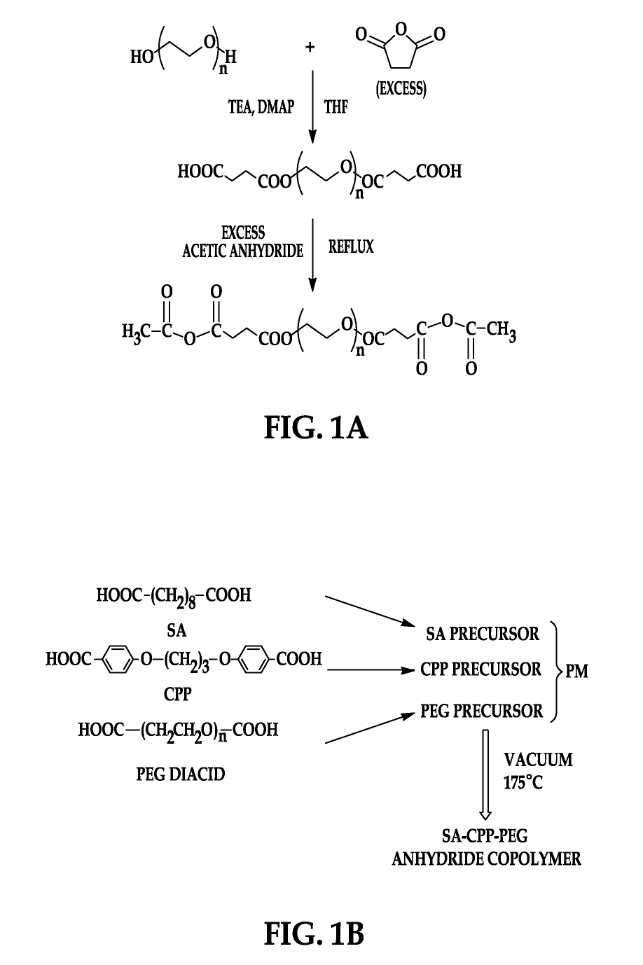

It is to be understood that the copolymers may have different molecular weight PEGs incorporated therein as hydrophilic segments. Generally, the poly(ethylene glycol) anhydride precursor may be formed from PEGs having a molecular weight ranging from about 100 to about 10,000 (e.g., PEG100 to PEG10,000). In an embodiment, the poly(ethylene glycol) anhydride precursor is formed from PEG600 having a molecular weight of about 600 and two carboxyl end groups or PEG1000 having a molecular weight about 1000 and two hydroxyl end groups. It is to be understood that, as depicted in FIG. 1A, the hydroxyl end groups of the PEG1000 are transformed into carboxylic groups before the anhydride precursor is formed. This reaction may be carried out through the catalysis of N,N′-dimethylamino-4-pyridine (DMAP), generally under mild conditions at room temperature.

FIG. 1A depicts the formation of PEG1000 (i.e., PEG diacid). Succinic anhydride and DMAP are substantially completely dissolved in anhydrous tetrahydrofuran (THF) and kept at 0° C. for a predetermined time. Poly(ethylene glycol) and triethylamine (TEA) are mixed in THF and transferred slowly into the succinic anhydride solution, generally under a nitrogen atmosphere. Once the reaction is complete, excess solvent is removed and the (PEG diacid) product is concentrated and precipitated. The PEG diacid may then be refluxed with acetic anhydride under nitrogen protection. Excess acetic anhydride is generally removed and the crude product of the PEG anhydride precursor is formed.

It is to be understood that the SA anhydride precursor and the CPP anhydride precursor may be synthesized using any suitable technique.

FIG. 1B generally depicts the formation of the PEG, SA, and CPP anhydride precursors from sebacic acid, 1,3-bis(carboxyphenoxy) propane, and poly(ethylene glycol). An embodiment of the method of forming the three-component polyanhydride copolymer includes mixing the precursors together to form a precursor mixture PM, as shown in FIG. 1B.

The precursor mixture PM is then melt polymerized to form the three-component polyanhydride copolymer. Melt polymerization of the precursors may be accomplished by heating the precursor mixture PM. As a non-limiting example, heating may be accomplished by immersing a container having the precursor mixture PM therein into a heated bath to initiate the formation of a reaction mixture. A non-limitative example of such a heated bath is an oil bath maintained at about 175° C. A vacuum atmosphere may be maintained in the container over the reaction mixture as the reaction mixture is stirred. The reaction mixture may also be purged with dry nitrogen for a predetermined time at predetermined time intervals as the reaction takes place.

It is to be understood that the three-component polyanhydride copolymer may be stored in a substantially non-hydrous environment at low temperature. It is to be understood that any suitable non-hydrous environment may be used, some examples of which include, but are not limited to hexane, other non-hydrous organic liquids, dried air, a vacuum, inert gas(es) (e.g., nitrogen gas), or combinations thereof. It is to be understood that the low temperature may be any temperature that is suitable or desired. In an embodiment, the low temperature ranges from about 4° C. to about −90° C. In a non-limitative embodiment, the three-component polyanhydride copolymer is stored in nitrogen at about −20° C.

To further illustrate embodiment(s) of the present disclosure, an example is given herein. It is to be understood that this example is provided for illustrative purposes and is not to be construed as limiting the scope of the disclosed embodiment(s).

### EXAMPLE

In this example, the following materials were used as received from Sigma-Aldrich Company: sebacic acid, 4-hydroxybenzoic acid, 1,3-dibromopropane, acetic anhydride, succinic anhydride, N,N′-dimethylamino-4-pyridine (DMAP), triethylamine (TEA), poly(ethylene glycol) (molecular weight: 1000 g/mol, PEG1000), poly(ethylene glycol)dicarboxy ether (molecular weight: 600, PEG600), tetrahydrofuran (THF), chloroform, sodium hydroxide, and diethyl ether. 1,3-(p-carboxyphenoxy)propane was synthesized and purified following the protocol published by A. Conix in Macromolecular Synthesis 1966; 2:95-99.

### PEG600 and Synthesis of PEG Diacid (PEG1000)

Two types of PEGs with molecular weights of 600 and 1000 (designated as PEG600 and PEG1000) were used to synthesize SA-CPP-PEG polyanhydrides. The PEG600 had two carboxyl end groups, while the PEG1000 had two hydroxyl end groups. Both hydroxyl end groups of the PEG1000 were first transformed into carboxylic groups before polymerization. This reaction was carried out under mild conditions at room temperature through the catalysis of N,N′-dimethylamino-4-pyridine (DMAP).

PEG diacid (PEG 1000) was synthesized by completely dissolving about 1.46 g/14.6 mmol succinic anhydride and about 209 mg/1.71 mmol DMAP in about 15 mL anhydrous THF. This solution was maintained at 0° C. for about 30 minutes. About 4.28 g/4.28 mmol poly(ethylene glycol) and about 1.8 mL/12.8 mmol triethyl amine were mixed in 15 ml THF and transferred slowly into the succinic anhydride solution through a syringe under a nitrogen atmosphere. The solution was stirred at 0° C. for about two hours. The reaction was allowed to continue at room temperature overnight. After the reaction was complete, the reaction solution was concentrated by removing most of the solvent. The PEG diacid was precipitated in cold diethyl ether. The precipitates were dissolved in dichloromethane again and then re-precipitated in cold diethyl ether. The precipitates were dried under vacuum at room temperature overnight.

### Synthesis of Anhydride Precursors

SA and CPP anhydride precursors were synthesized according to procedures described by A. J. Domb and R. Langer in the article entitled “Polyanhydrides .1. Preparation of High-Molecular-Weight Polyanhydrides” published in J. Polym. Sci. Pol. Chem. In 1987; 25(12):33 at pages 73-3386.

About 4 g/3.3 mmol PEG diacid was refluxed with about 20 mL/210 mmol acetic anhydride for about 4 hours under nitrogen protection. Excess acetic anhydride was removed by distillation under vacuum. The crude product of the PEG anhydride precursor was washed three times with diethyl ether, and the precipitate was dried under vacuum at room temperature overnight.

### Synthesis of Polyanhydrides

SA, CCP and PEG anhydride precursors were charged into a pre-dried Schlenk tube. Alternating cycles of vacuum and nitrogen purging were repeated three times, and the vacuum was maintained inside the tube at the end. The Schlenk tube was then immersed into an oil bath and the vacuum was maintained under continuous pumping. The reaction mixture was stirred vigorously and purged with dry nitrogen for about 30 seconds every fifteen minutes. At the end of the reaction, the resulting polymer melt was stored in a vial filled with nitrogen at about −20° C.

### Structural Characterization

NMR spectra were recorded at room temperature using a Varian 300 NMR spectrometer with the residual normal solvent in the deuterated solvent as the reference. Compressive modulus was determined at room temperature using MTS Synergie 200 mechanical tester (MTS Systems Corporation, Eden Prairie, Minn.). Cylinder-shaped specimens with dimensions of 3.55 mm in diameter and 2 mm in thickness were punched out of the resulting polymer melt and were cooled to room temperature in a desiccator under lab-bench vacuum. The crosshead speed was about 0.5 mm/min and the compressive modulus was defined as the initial linear modulus.

The molecular weights were calculated using a standard intrinsic viscosity measurement. The viscosity measurement was conducted at 23° C. in an Ubbelohde viscometer. Afflux times of the polyanhydride solutions were measured at five concentrations, using anhydrous chloroform as the solvent. These afflux times were used to determine the relative viscosities and the intrinsic viscosity.

Scanning electron microscopy was used to observe the morphology of degrading polymer specimens with an accelerating voltage of 10 kV. The specimens were coated with gold for 130 seconds using a sputter coater. During the coating process, the gas pressure was maintained at about 50 mtorr and the current maintained at about 40 mA.

### Discussion of Synthesis of SA-CPP-PEG Three-Component Polyanhydrides

To focus on the effects of PEG in the anhydride polymer, the ratio between SA and CPP in the polyanhydrides was kept at 80:20. The feeding of PEG was varied from about 1% to about 10% with respect to the total molar amounts of SA and CPP. FIG. 2 shows the NMR spectrum of a poly(SA-CPP-PEG) anhydride. The peaks corresponding to PEG (3.4-3.8 ppm), CPP (6.9 and 8.0 ppm), and SA (1.4-1.8 ppm) confirmed the presence of the three components in the newly synthesized polyanhydrides. The PEG content in polyanhydrides was obtained by calculating the ratio of integrations of corresponding NMR peaks.

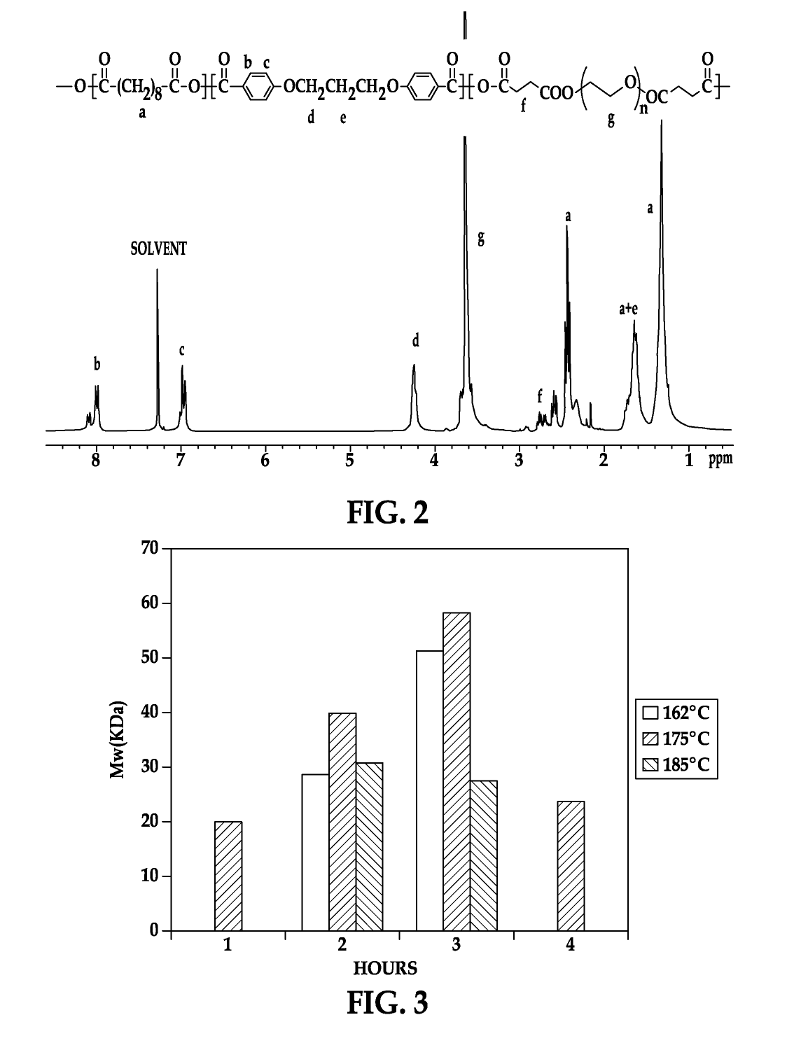

The reaction conditions were optimized by varying the reaction temperature and the reaction time. As shown in FIG. 3, the highest molecular weight product was prepared at about 175° C. for about three hours. During the reaction, the viscosity of the reaction mixture gradually increased and the color of the melt changed from white to light yellow. The polymerization yield ranged from about 72% to about 84%.

Table 1 (below) depicts the contents of PEG in the final products, the intrinsic viscosity ([η]), molecular weight (M), the yield, the compressive modulus, and the contact angle.

TABLE 1
Properties of synthesized SA-CPP-PEG polyanhydrides

| | | PEG % | PEG % | [η] | | | Modulus | Contact |
|---|---|---|---|---|---|---|---|---|
| Sample | PEG | (mol.)^{a} | (mol.)^{b} | (dl/g)^{c} | Mw^{d} × 10⁻³ | Yield % | (MPa)^{e} | angle |
| A1 | 600 | 1 | 1.1 | 0.533 | 58.8 | 72 | 58.1 | 69.3 |
| A2 | 600 | 2.5 | 2.6 | 0.513 | 55.5 | 79 | 52.1 | 64.2 |
| A3 | 600 | 5.0 | 5.3 | 0.385 | 35.9 | 76 | 25.6 | 51.6 |
| A4 | 600 | 10.0 | 10.4 | 0.382 | 35.5 | 73 | 7.13 | 36.9 |
| B1 | 1000 | 1 | 1.1 | 0.296 | 24.1 | 73 | 58.5 | 62.2 |
| B2 | 1000 | 2.5 | 2.8 | 0.308 | 25.6 | 84 | 54.3 | 54.6 |
| B3 | 1000 | 5.0 | 5.7 | 0.316 | 26.6 | 78 | 22.6 | 41.4 |
| B4 | 1000 | 10.0 | 11.9 | 0.199 | 13.2 | 79 | 2.24 | 35.8 |
| C | — | — | — | 0.497 | 52.9 | 68 | 64.2 | 89.6 |

^{a}Feeding molar ratio of PEG to the sum of SA and CPP
^{b}PEG content in polyanydride measured by ¹H-NMR
^{c}Measured in chloroform solution at 23° C.
^{d}Calculated based on equation [η] = 3.88 × 10⁻⁷Mw^{0.658}
^{e}Compressive modulus measured at room temperature

The results show that the PEG content in the final products is relatively close to the feeding percentages, which indicates that the PEG precursors have similar reactivity to SA and CPP precursors. Therefore, these three components are likely arranged with random sequences in the polyanhydrides.

Since there is a lack of relationship between the intrinsic viscosity ([η]) and molecular weight (M) of the three-component polyanhydrides (containing PEG), the [η]-M relationship of two-component (SA-CPP) polyanhydrides (no PEG) was used to estimate the molecular weights, for relative comparison purpose. It was found that, at the same PEG content, the intrinsic viscosity of PEG-containing polyanhydrides with PEG600 was higher than the intrinsic viscosity of those containing PEG1000. This may have resulted from a higher molecular weight of the PEG600-containing polyanhydrides, and/or from the higher chain flexibility of the PEG1000 in a polyanhydride with a similar molecular weight to that of the PEG600-containing polyanhydride.

As previously stated, compressive modulus and contact angle measurements are also listed in Table 1. As the PEG content increased, the compressive modulus decreased. When the PEG content reached about 10%, the polyanhydrides became wax-like materials and the compressive modulus decreased significantly. The contact angle also decreased as the PEG content increased, which indicated an increase in hydrophilicity. During contact angle measurements, it was observed that a relatively tiny drop of water gradually expanded on the polymer surface when PEG content was higher (˜10%). This is an indication that an increase in hydrophilicity may be attributed to the introduction of the PEG segments into the polyanhydrides.

### Erosion Behavior of Poly(SA-CPP-PEG)

Erosion experiments of the synthesized polyanhydrides were conducted by measuring the mass loss of specimens and pH value change of the PBS solutions. The erosion experiments were conducted in a 0.1 M phosphate buffer solution (PBS) at 37° C. Cylinder-shaped specimens with a diameter of 3.55 mm and three different thicknesses (1 mm, 0.50 mm and 0.25 mm) were prepared, except for polyanhydrides containing about 10% PEG. When PEG content was about 10%, the polyanhydrides became very hydroscopic and too soft to prepare the specimens. During the erosion experiments, most specimens with a thickness of 0.25 mm broke when removal of the PBS was attempted, and therefore the mass loss was not obtained.

The specimens were weighed before they were immersed into PBS. The buffer solution was renewed every 24 hours. The specimens were removed at designated times and dried in a vacuum oven at room temperature overnight. The specimens were weighed again to obtain the mass loss from the erosion.

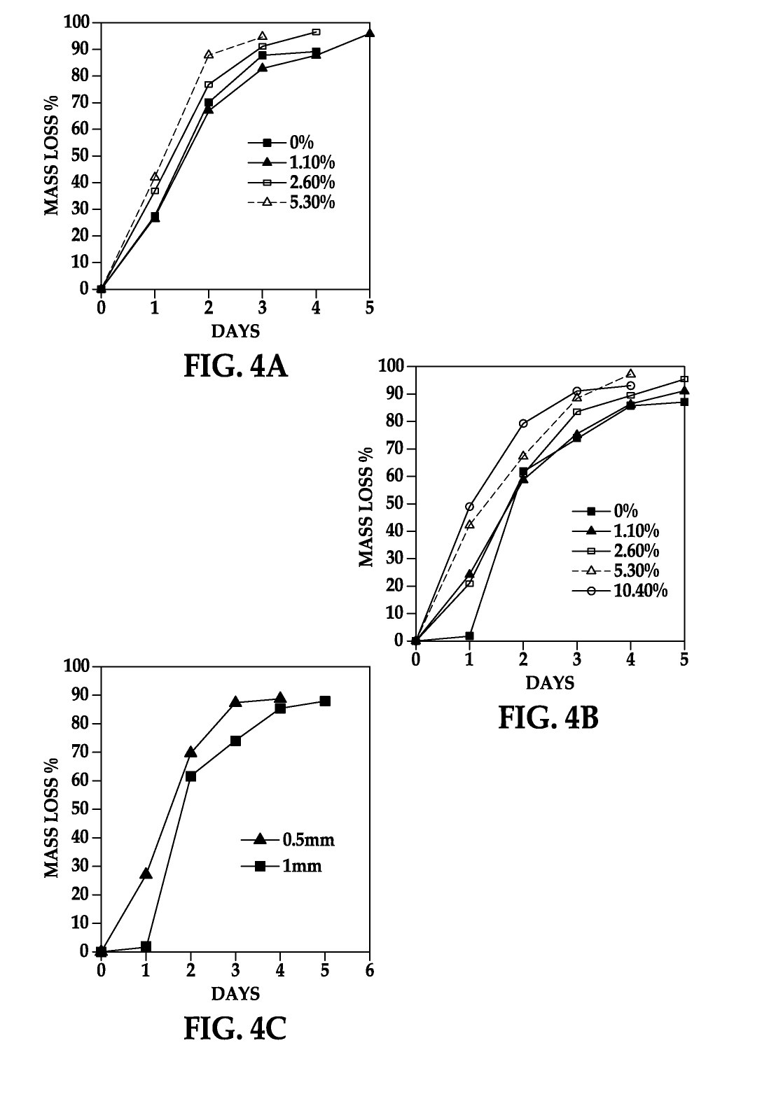

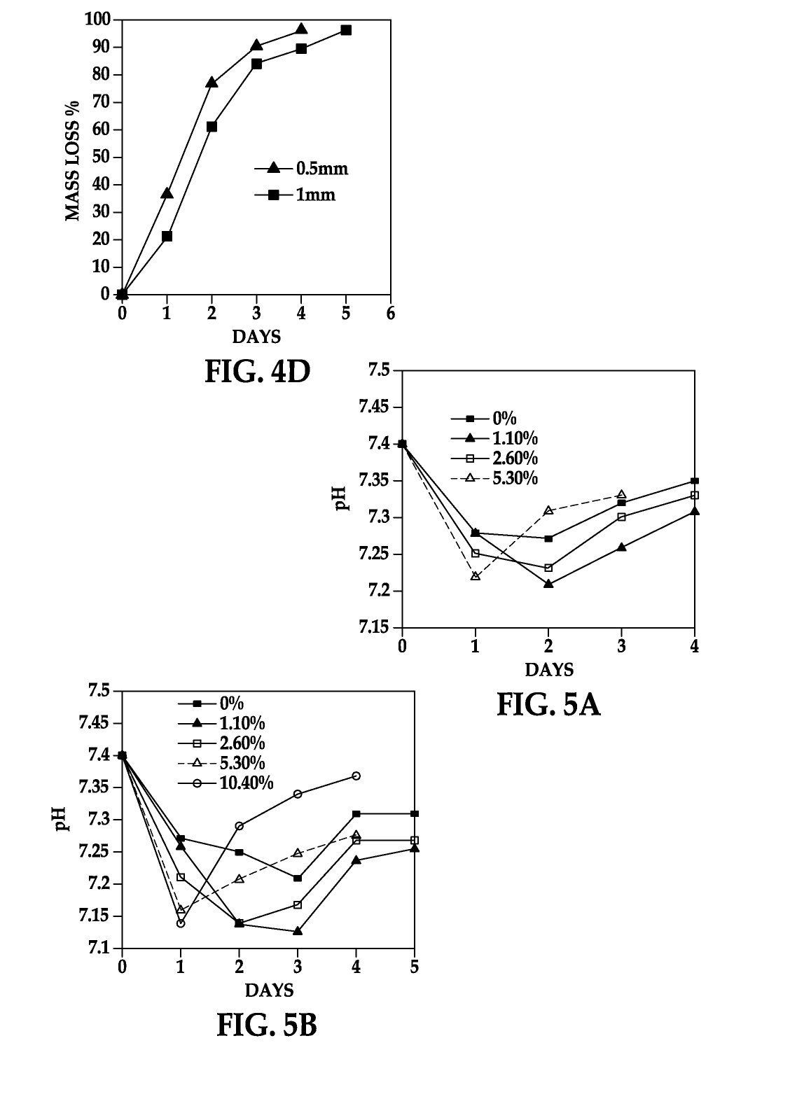

FIGS. 4A through 4D show the erosion profiles based on mass loss data. After 4 days of erosion, the cumulative percentage mass loss was over 80% for all of the three-component polyanhydrides. FIGS. 4A and 4B depict erosion profiles of specimens with the same thickness. Comparing these figures shows that the specimens with higher PEG contents had faster erosion rates. The sample did become very fragile once the mass loss was over 80% and reached the plateau region. This was associated with the completion of erosion. The early plateau on the mass loss curve indicated that the erosion process reached completion in a short period of time. This faster erosion characteristic is desirable for PTH delivery profiles.

Assuming a substantially solid cylinder shape, the calculations showed that the surface areas for the specimens 1.0 mm and 0.5 mm in thickness are 30.9 mm² and 25.4 mm², respectively. The corresponding volumes are 9.9 mm³ and 4.95 mm³ respectively, which are proportional to the masses of the specimens. The surface/volume ratios for specimens 1.0 mm and 0.5 mm in thickness are 3.1 mm⁻¹ and 5.1 mm⁻¹, respectively. It is believed that for surface erosion materials, the specimens with larger surface/volume ratio should erode faster. FIGS. 4C and 4D depict the erosion rates for the same polyanhydrides with different thicknesses. For both SA-CPP (FIG. 4C) and SA-CPP-PEG (FIG. 4D) polyanhydrides, the thinner specimens (with higher surface/volume ratio) erode faster. The polyanhydride containing PEG (2.6% PEG600) shows a similar mass loss behavior to that of the polyanhydride without PEG in this regard. This indicates that the polyanhydrides containing PEG retain the surface erosion properties.

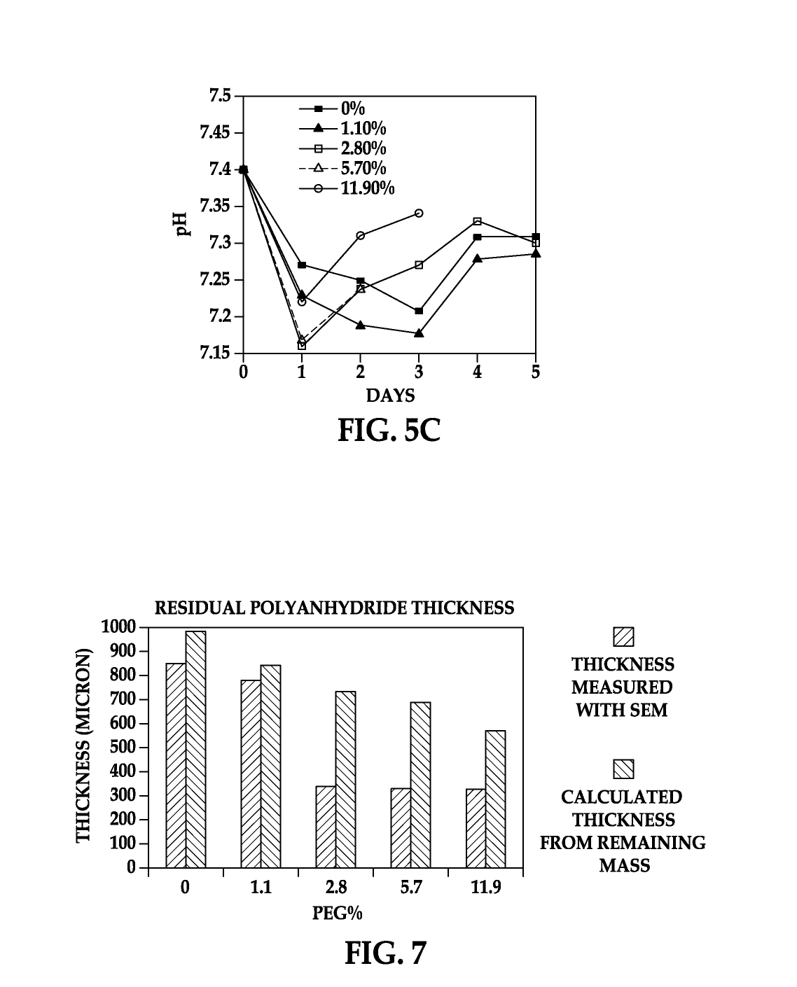

The pH values of the solutions were determined at room temperature using a pH meter calibrated with three standard solutions. As depicted in FIGS. 5A through 5C, the pH value of the PBS solutions during the erosion was always higher than 7.10. This property may be favorable for minimizing potential inflammation when used with in vivo applications. For the 1 mm thick specimens (FIG. 5B), the pH value reached a minimum after three days of erosion, and for the 0.50 mm thick specimens (FIG. 5A), the pH value reached a minimum after two days of erosion. The polyanhydrides with PEG content more than about 5% showed the minimum pH just after one day of erosion. These results demonstrate that the acceleration of erosion resulted from the increased PEG content. These results also strengthen the notion that the thinner specimens eroded faster, which is generally characteristic of surface erosion materials.

As shown in FIG. 5C (PEG1000 in the polyanhydrides), the minimum pH value was reached after three days of erosion when the PEG content was about 1.1%. The minimum was reached one day into the erosion when the PEG content ranged from about 2.8% to about 11.9%. Considering that PEG1000 was more hydrophilic than PEG600, which was confirmed by contact angle measurements, the erosion of polyanhydrides containing PEG1000 should be faster than that of polyanhydrides containing PEG600. This was supported by the data that polyanhydride containing 2.8% PEG1000 reached the minimum pH value after one day of erosion while the polyanhydride containing 2.6% PEG600 reached the minimum pH value after two days of erosion (compare FIGS. 5B and 5C).

### SEM Observation

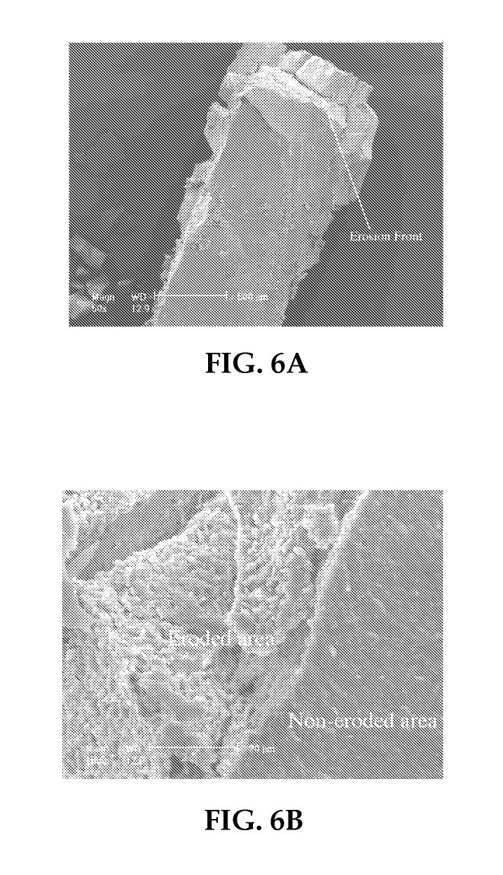

While the mass loss data from the erosion experiments revealed surface erosion characteristics, they do not provide a direct image of surface erosion. Scanning electron microscopy (SEM) was used to visualize and to study the morphology of the polymer materials at different stages of the erosion process. The SEM images (FIGS. 6A and 6B) in general are able to signify the moving erosion front, which is a typical indication of surface erosion. The porous materials formed on the surface of specimens illustrate a clear contrast between the eroded part and non-eroded part, which allows for estimation of the residual thickness of polyanhydrides at different erosion stages.

In FIGS. 6A and 6B, there is a clear erosion front between the eroded portions and the remaining specimen. The eroded portions were so fragile that they often fell off when the SEM specimens were prepared. It was found that polyanhydride samples containing PEG exhibited faster erosion progression compared to samples without PEG. This result corroborated with those of mass loss and pH value measurements, indicating that addition of PEG facilitated the erosion of polyanhydrides. The thicknesses of residual polyanhydride observed from SEM and those calculated from the remaining mass are compared in FIG. 7. At higher PEG contents in the polyanhydrides (greater than about 1.1%), the residual thickness was significantly smaller after erosion. The thicknesses of polyanhydride specimens measured from SEM photos were smaller than those calculated from mass loss percentage, in part because, the portions of eroded materials still contributed to the remaining mass. The PEG-containing polyanhydride specimens became substantially completely porous after two days of erosion, with an exception of polyanhydride containing about 1.1% PEG.

It is to be understood that a device incorporating embodiments of the three-component polyanhydride copolymer may be suitable for releasing parathyroid hormone, and other drugs, hormones, etc., in a pulsatile fashion. The device generally includes a plurality of biodegradable polymer layers. Each of the biodegradable polymer layers includes an embodiment of the three-component polyanhydride copolymer (i.e., SA-CPP-PEG anhydride copolymer). Between each of the plurality of biodegradable polymer layers is a layer of parathyroid hormone (or other suitable drugs, including but not limited to pulsatile released drugs, antibiotics, and/or the like, and/or combinations thereof). In an embodiment, the layer of hormone/drug/etc. is mixed in a water soluble matrix containing the hormone/drug/etc. It is to be understood that the matrix may be any suitable water soluble matrix, including, but not limited to synthetic or natural matrices, large or small molecule matrices, and/or combinations thereof.

The device may be configured so that as and/or after the outermost biodegradable polymer layer decomposes/erodes, the parathyroid hormone layer is exposed and may be released. As such, the degradation of the biodegradable polymer and release of the parathyroid hormone occur intermittently.

The three-component polyanhydrides described herein include, but are not limited to the following advantages. In addition to the device disclosed herein, the copolymers may also be suitable for use in, for example, tissue engineering scaffold molds, temporary separation materials, or the like. The surface erosion characteristics of polyanhydrides may advantageously be retained when the PEG content is low. It has been found that higher PEG content increases the hydrophilicity and erosion rate. As such, the PEG content may be varied to tailor the erosion rate of the polyanhydrides, which may be advantageous in devices for pulsatile drug release.

While several embodiments have been described in detail, it will be apparent to those skilled in the art that the disclosed embodiments may be modified. Therefore, the foregoing description is to be considered exemplary rather than limiting.

What is claimed is:

1. A three-component polyanhydride copolymer having tunable erosion properties, comprising:
a sebacic acid anhydride precursor;
a 1,3-bis(carboxyphenoxy) propane anhydride precursor; and
a poly(ethylene glycol) anhydride precursor;
wherein an erosion rate of the copolymer increases with an increasing amount of the poly(ethylene glycol) anhydride precursor.

2. The three-component polyanhydride copolymer as defined in claim 1 wherein a ratio of the sebacic acid anhydride precursor to the 1,3-bis(carboxyphenoxy) propane anhydride precursor ranges from about 95:5 to about 5:95.

3. The three-component polyanhydride copolymer as defined in claim 1 wherein a ratio of the sebacic acid anhydride precursor to the 1,3-bis(carboxyphenoxy) propane anhydride precursor is about 80:20.

4. The three-component polyanhydride copolymer as defined in claim 1 wherein the poly(ethylene glycol) anhydride precursor is present in an amount ranging from about 1% to about 25% with respect to a total molar amount of the sebacic acid anhydride precursor and the 1,3-bis(carboxyphenoxy) propane anhydride precursor.

5. The three-component polyanhydride copolymer as defined in claim 1 wherein hydrophilicity of the copolymer increases with an increasing amount of the poly(ethylene glycol) anhydride precursor.

6. The three-component polyanhydride copolymer as defined in claim 1 wherein the poly(ethylene glycol) anhydride precursor is formed from polyethylene glycol having a molecular weight ranging from about 100 to about 10,000.

7. The three-component polyanhydride copolymer as defined in claim 6 wherein the poly(ethylene glycol) anhydride precursor is formed from polyethylene glycol having a molecular weight of about 600, and having two carboxyl end groups.

8. The three-component polyanhydride copolymer as defined in claim 6 wherein the poly(ethylene glycol) anhydride precursor is formed from polyethylene glycol having a molecular weight of about 1000, and having two hydroxyl end groups.

9. A method for forming a three-component polyanhydride copolymer, comprising:
combining a sebacic acid anhydride precursor, a 1,3-bis(carboxyphenoxy) propane anhydride precursor, and a poly(ethylene glycol) anhydride precursor, thereby forming a precursor mixture; and
melt polymerizing the precursor mixture to form the three-component polyanhydride copolymer.

10. The method as defined in claim 9 where prior to combining, the method further comprises:
forming poly(ethylene glycol) diacid;
refluxing a predetermined amount of the poly(ethylene glycol) diacid with a predetermined amount of acetic anhydride for a predetermined time; and
removing excess acetic anhydride, thereby forming the poly(ethylene glycol) anhydride precursor.

11. The method as defined in claim 9 wherein melt processing is accomplished by:
heating the precursor mixture, thereby forming a reaction mixture;
maintaining a vacuum over the reaction mixture; and
purging the reaction mixture with dry nitrogen for a predetermined time.

12. The method as defined in claim 11 wherein heating the precursor mixture is accomplished by immersing a container having the precursor mixture therein into a heated bath.

13. The method as defined in claim 9, further comprising storing the three-component polyanhydride copolymer in a low temperature environment and a substantially non-hydrous environment.

14. The method as defined in claim 13 wherein the low temperature environment ranges from about 4° C. to about −90° C.

15. The method as defined in claim 13 wherein the substantially non-hydrous environment is selected from non-hydrous organic liquids, dried air, inert gases, and combinations thereof.

16. A device for releasing parathyroid hormone, comprising:
a plurality of biodegradable polymer layers, each of the biodegradable polymer layers including a copolymer of a sebacic acid anhydride precursor, a 1,3-bis(carboxyphenoxy) propane anhydride precursor, and a poly(ethylene glycol) anhydride precursor, wherein a degradation rate of the copolymer increases within increasing amount of the poly(ethylene glycol) anhydride precursor; and
a layer of parathyroid hormone established between each of the plurality of biodegradable polymer layers, whereby degradation of the biodegradable polymer and release of the parathyroid hormone occur intermittently.

17. The device as defined in claim 16 wherein the plurality of biodegradable polymer layers is configured to have a predetermined degradation rate so that the parathyroid hormone is released in a pulsatile fashion.
